(19) United States
(12) Patent Application Publication
(10) Pub. No.: US 2017/0323130 A1
(43) Pub. Date: Nov. 9, 2017

(54) BIDIRECTIONAL ULTRASONIC SENSOR SYSTEM FOR BIOMETRIC DEVICES

(71) Applicant: QUALCOMM Incorporated, San Diego, CA (US)

(72) Inventors: Timothy Alan Dickinson, Carlsbad, CA (US); Nicholas Ian Buchan, San Jose, CA (US); David William Burns, San Jose, CA (US); John Keith Schneider, Williamsville, NY (US); Muhammed Ibrahim Sezan, Los Gatos, CA (US)

(21) Appl. No.: 15/149,043

(22) Filed: May 6, 2016

(51) Int. Cl. G06K 9/00; G06F 21/32; A61B 8/00; A61B 8/08; A61B 8/14; A61B 8/02

(52) U.S. Cl. CPC G06K 9/0002; G06K 9/0012; G06K 9/00026; G06F 21/32; A61B 8/56; A61B 8/02; A61B 8/5207; A61B 8/4236; A61B 8/4227; A61B 8/145; A61B 8/54

(57) ABSTRACT

An apparatus may include an ultrasonic receiver array, an ultrasonic transmitter and a control system capable of controlling the ultrasonic transmitter to transmit first ultrasonic waves in a first direction and to simultaneously transmit second ultrasonic waves in a second direction that is opposite the first direction. The control system may be capable of distinguishing first reflected waves from second reflected waves, the first reflected waves corresponding to reflections of the first ultrasonic waves that are received by the ultrasonic receiver array and the second reflected waves corresponding to reflections of the second ultrasonic waves that are received by the ultrasonic receiver array. The control system may be capable of determining first image data corresponding to the first reflected waves and of determining second image data corresponding to the second reflected waves.

### TECHNICAL FIELD

This disclosure relates generally to biometric devices and methods, particularly biometric devices and methods applicable to mobile devices, including but not limited to wearable devices.

### DESCRIPTION OF THE RELATED TECHNOLOGY

As mobile devices become more versatile, user authentication becomes increasingly important. Increasing amounts of personal information may be stored on and/or accessible by a mobile device. Moreover, mobile devices are increasingly being used to make purchases and perform other commercial transactions. Some mobile devices, including but not limited to wearable devices, currently include fingerprint sensors for user authentication. Improved authentication methods would be desirable.

### SUMMARY

The systems, methods and devices of the disclosure each have several innovative aspects, no single one of which is solely responsible for the desirable attributes disclosed herein.

One innovative aspect of the subject matter described in this disclosure can be implemented in an apparatus. The apparatus may include an ultrasonic receiver array, an ultrasonic transmitter and a control system. The control system may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof.

The control system may be capable of controlling the ultrasonic transmitter to transmit first ultrasonic waves in a first direction and to simultaneously transmit second ultrasonic waves in a second direction that is opposite the first direction. The control system may be capable of distinguishing first reflected waves from second reflected waves. The first reflected waves may correspond to reflections of the first ultrasonic waves that are received by the ultrasonic receiver array and the second reflected waves may correspond to reflections of the second ultrasonic waves that are received by the ultrasonic receiver array. The control system may be capable of determining first image data corresponding to the first reflected waves and of determining second image data corresponding to the second reflected waves.

In some examples, the first reflected waves may be received at the ultrasonic receiver array from a direction that is opposite a direction from which the second reflected waves are received. According to some implementations, the control system may be capable of distinguishing the first reflected waves from the second reflected waves based on a selected range-gate delay, frequency-dependent content of the first reflected waves and the second reflected waves, one or more image processing methods and/or temperature data received from at least one temperature sensor.

According to some examples, the control system may be capable of performing an authentication process, a liveness determination process and/or a pulse rate detection process. These processes may be based, at least in part, on the first image data, on the second image data or on both the first image data and the second image data. In some examples, the first image data, the second image data, or both the first image data and the second image data may include fingerprint image data. In some instances, the first image data may include image data corresponding to a finger of a user and the second image data may include image data corresponding to a thumb of the user.

In some implementations, the control system may be capable of detecting motion according to changes in at least one of the first image data or the second image data. In some such implementations, the control system may be capable of producing a motion-detection signal that corresponds with a detected motion. The apparatus may, in some examples, include a wireless interface. In some such implementations, the control system may be capable of transmitting the motion-detection signal via the wireless interface.

According to some implementations, a wearable device may be, or may include, the apparatus. For example, the wearable device may be a bracelet, an armband, a wristband, a ring, a headband or a patch. In some implementations, the control system may be capable of obtaining at least one of a tissue image or a bone image from a first side of the wearable device. According to some such implementations, the control system may be capable of obtaining a fingerprint image from a second side of the wearable device. In some examples, the control system may be capable of performing an authentication process, a liveness determination process and/or a pulse rate detection process. The process or processes may be based, at least in part, on images obtained from the first side, from the second side, or from both the first side and the second side of the wearable device.

In some examples, a smart card may be, or may include, the apparatus. According to some such examples, the control system may be capable of communication with a smart card reader. According to some implementations, the control system may be capable of performing an authentication process according to the first image data and the second image data. In some such implementations, the first image data, the second image data, or both the first image data and the second image data may include fingerprint image data. However, in some examples, the first image data, the second image data, or both the first image data and the second image data may include image data corresponding to a thumb.

Other innovative aspects of the subject matter described in this disclosure can be implemented in a method of controlling a biometric sensor system that may involve controlling an ultrasonic transmitter to transmit first ultrasonic waves in a first direction and to simultaneously transmit second ultrasonic waves in a second direction that is opposite the first direction. The method may involve distinguishing first reflected waves from second reflected waves. The first reflected waves may correspond to reflections of the first ultrasonic waves that are received by an ultrasonic receiver array and the second reflected waves may correspond to reflections of the second ultrasonic waves that are received by the ultrasonic receiver array. The method may involve determining first image data corresponding to the first reflected waves. The method may involve determining second image data corresponding to the second reflected waves.

In some examples, the first reflected waves may be received at the ultrasonic receiver array from a direction that is opposite a direction from which the second reflected waves are received. According to some implementations, distinguishing the first reflected waves from the second reflected waves may be based on a selected range-gate delay, frequency-dependent content of the first reflected waves and the second reflected waves, one or more image processing methods and/or temperature data received from at least one temperature sensor of the biometric sensor system.

In some examples, the method may involve an authentication process, a liveness determination process or a pulse rate detection process according to the first image data, the second image data, or both the first image data and the second image data. According to some such examples, the first image data, the second image data, or both the first image data and the second image data may include fingerprint image data.

Some or all of the methods described herein may be performed by one or more devices according to instructions (e.g., software) stored on non-transitory media. Such non-transitory media may include memory devices such as those described herein, including but not limited to random access memory (RAM) devices, read-only memory (ROM) devices, etc. Accordingly, some innovative aspects of the subject matter described in this disclosure can be implemented in a non-transitory medium having software stored thereon.

For example, the software may include instructions for controlling an ultrasonic transmitter to transmit first ultrasonic waves in a first direction and to simultaneously transmit second ultrasonic waves in a second direction that is opposite the first direction. The software may include instructions for controlling a control system to distinguish first reflected waves from second reflected waves. The first reflected waves may correspond to reflections of the first ultrasonic waves that are received by an ultrasonic receiver array and the second reflected waves may correspond to reflections of the second ultrasonic waves that are received by the ultrasonic receiver array.

The software may include instructions for controlling the control system to determine first image data corresponding to the first reflected waves. The software may include instructions for controlling the control system to determine second image data corresponding to the second reflected waves.

In some examples, the first reflected waves may be received at the ultrasonic receiver array from a direction that is opposite a direction from which the second reflected waves are received.

In some examples, the software may include instructions for controlling the control system to distinguish the first reflected waves from the second reflected waves based on a selected range-gate delay, frequency-dependent content of the first reflected waves and the second reflected waves, one or more image processing methods and/or temperature data received from at least one temperature sensor of the biometric sensor system.

In some examples, the software may include instructions for controlling the control system to perform an authentication process, a liveness determination process and/or a pulse rate detection process. The process(es) may be based, at least in part, on the first image data, on the second image data, or on the first image data and the second image data. According to some examples, the first image data, the second image data, or both the first image data and the second image data may include fingerprint image data.

Still other innovative aspects of the subject matter described in this disclosure can be implemented in a smart card that includes an ultrasonic receiver array, an ultrasonic transmitter and a control system. The control system may be capable of controlling the ultrasonic transmitter to transmit first ultrasonic waves in a first direction and to simultaneously transmit second ultrasonic waves in a second direction that is opposite the first direction. The first direction may be towards a first side of the smart card and the second direction may be towards a second side of the smart card.

The control system may be capable of distinguishing first reflected waves from second reflected waves. The first reflected waves may correspond to reflections of the first ultrasonic waves that are received by the ultrasonic receiver array and the second reflected waves may correspond to reflections of the second ultrasonic waves that are received by the ultrasonic receiver array. The control system may be capable of determining first image data corresponding to the first reflected waves and of determining second image data corresponding to the second reflected waves.

In some examples, the control system may be capable of communication with a smart card reader. In some implementations, the control system may be capable of performing an authentication process, a liveness determination process or a pulse rate detection process. The process(es) may be based, at least in part, on the first image data, on the second image data, or on the first image data and the second image data. According to some examples, the first image data, the second image data, or both the first image data and the second image data may include fingerprint image data. In some instances, the first image data, the second image data, or both the first image data and the second image data may include image data corresponding to a thumb.

### BRIEF DESCRIPTION OF THE DRAWINGS

Details of one or more implementations of the subject matter described in this specification are set forth in the accompanying drawings and the description below. Other features, aspects, and advantages will become apparent from the description, the drawings, and the claims. Note that the relative dimensions of the following figures may not be drawn to scale. Like reference numbers and designations in the various drawings indicate like elements.

FIG. 1 is a block diagram that shows example components of an apparatus according to some implementations.

FIG. 2 is a flow diagram that provides examples of biometric sensor system operations.

FIG. 3A shows an example of a cross-sectional view of an ultrasonic sensor system capable of performing the method of FIG. 2.

FIG. 3B indicates the distances traveled by the ultrasonic waves shown in FIG. 3A, from the time of transmission by the ultrasonic transmitter until the time of receipt by the ultrasonic receiver array.

FIG. 4 includes two diagrams that illustrate an example of distinguishing first reflected waves from second reflected waves according to acquisition time delays.

FIG. 5 shows an example of a headband that includes a biometric sensor system.

FIG. 6 shows an example of a patch that includes a biometric sensor system.

FIG. 7 shows an example of a wristband that includes a biometric sensor system.

FIG. 8 shows an example of an armband that includes a biometric sensor system.

FIGS. 9A and 9B show examples of rings that include biometric sensor systems.

FIG. 10 shows an example of a key fob that includes a biometric sensor system.

FIGS. 11A-11C provide examples of detecting motion according to changes in fingerprint image data or thumbprint image data.

FIG. 12 shows examples of wrist image features.

FIG. 13A shows an example of a smart card that includes a biometric sensor system.

FIG. 13B shows an example of using the smart card of FIG. 13A.

FIG. 14 shows an example of pulse and/or liveness detection with a biometric sensor system.

FIG. 15A shows an example of an exploded view of an ultrasonic sensor system.

FIG. 15B shows an exploded view of an alternative example of an ultrasonic sensor system.

### DETAILED DESCRIPTION

The following description is directed to certain implementations for the purposes of describing the innovative aspects of this disclosure. However, a person having ordinary skill in the art will readily recognize that the teachings herein may be applied in a multitude of different ways. The described implementations may be implemented in any device, apparatus, or system that includes a biometric sensor system. In addition, it is contemplated that the described implementations may be included in or associated with a variety of electronic devices such as, but not limited to: mobile telephones, multimedia Internet enabled cellular telephones, mobile television receivers, wireless devices, smartphones, smart cards, wearable devices such as bracelets, armbands, wristbands, rings, headbands, patches, etc., Bluetooth® devices, personal data assistants (PDAs), wireless electronic mail receivers, hand-held or portable computers, netbooks, notebooks, smartbooks, tablets, printers, copiers, scanners, facsimile devices, global positioning system (GPS) receivers/navigators, cameras, digital media players (such as MP3 players), camcorders, game consoles, wrist watches, clocks, calculators, television monitors, flat panel displays, electronic reading devices (e.g., e-readers), mobile health devices, computer monitors, auto displays (including odometer and speedometer displays, etc.), cockpit controls and/or displays, camera view displays (such as the display of a rear view camera in a vehicle), electronic photographs, electronic billboards or signs, projectors, architectural structures, microwaves, refrigerators, stereo systems, cassette recorders or players, DVD players, CD players, VCRs, radios, portable memory chips, washers, dryers, washer/dryers, parking meters, packaging (such as in electromechanical systems (EMS) applications including microelectromechanical systems (MEMS) applications, as well as non-EMS applications), aesthetic structures (such as display of images on a piece of jewelry or clothing) and a variety of EMS devices. The teachings herein also may be used in applications such as, but not limited to, electronic switching devices, radio frequency filters, sensors, accelerometers, gyroscopes, motion-sensing devices, magnetometers, inertial components for consumer electronics, parts of consumer electronics products, varactors, liquid crystal devices, electrophoretic devices, drive schemes, manufacturing processes and electronic test equipment. Thus, the teachings are not intended to be limited to the implementations depicted solely in the Figures, but instead have wide applicability as will be readily apparent to one having ordinary skill in the art.

Various implementations disclosed herein may include ultrasonic sensor systems that can be used to image fingerprints. Some ultrasonic sensor systems may be capable of acquiring images of subcutaneous tissue. For example, the images may be planar scans or 3-D images of subcutaneous tissue. In some implementations, a biometric sensor system may include an ultrasonic sensor system having an ultrasonic receiver array and an ultrasonic transmitter. In some implementations, the ultrasonic transmitter may be included as part of the ultrasonic receiver array (e.g., a single-layer transmitter and receiver). The ultrasonic transmitter may be capable of transmitting first ultrasonic waves in a first direction and second ultrasonic waves in a second direction that is opposite, or substantially opposite, the first direction. In some examples, the ultrasonic transmitter may be capable of transmitting the first and second ultrasonic waves at the same time, or at substantially the same time. In some examples, the first direction may be away from the ultrasonic receiver array and the second direction may be towards the ultrasonic receiver array. Such directions may sometimes be referred to herein as “downward” and “upward” for the sake of convenience, although the actual orientations may vary according to implementation and usage. By distinguishing reflections of the downward and upward waves, ultrasonic images may be obtained from below the biometric sensor system, from above the biometric sensor system, or from both above and below the biometric sensor system.

Particular implementations of the subject matter described in this disclosure can be implemented to realize one or more of the following potential advantages. In some implementations, the ultrasonic sensor system may be thin enough for convenient use in a smart card, ring, armband, wristband, headband or skin patch. Some such implementations may be capable of imaging underlying tissue on a continuous or quasi-continuous basis. Some implementations may be capable of verifying or authenticating a fingerprint from a user when desired. In some implementations, the ultrasonic sensor system may be flexible, which may be a desirable property for implementation in a flexible armband, a ring, etc. In some implementations, the ultrasonic sensor system may be curved and rigidly affixed to a curved, rigid armband or ring. Some disclosed biometric sensor systems may include a bidirectional authenticating sensor array that is suitable for “pinch authentication” based on images of a user's thumb on one side of the biometric sensor system and a user's finger on another side of the biometric sensor system. Some implementations may include a bidirectional ultrasonic sensor system configurable for liveness determination and/or pulse rate (e.g. heartrate) detection.

Some implementations may be capable of gesture detection, e.g., by determining that a wearable device has been moved relative to a corresponding part of a user's body. For example, in some implementations a control system may be capable of determining whether a ring has been rotated or translated along a user's finger. In some such implementations, detected gestures may be forms of user input. Alternatively, gestures may be detected on one side of the biometric sensor while the other side remains in contact with a user to provide continuous or quasi-continuous authentication. Different types of gestures may correspond with different functionality desired by a user. Such functionality may involve control of another device with which the biometric sensor system is capable of communicating, e.g., via wireless communication. Accordingly, in some implementations the control system may be capable of detecting a motion of the biometric sensor system, or of an object positioned on or near the biometric sensor system, and of producing a signal that corresponds with the detected motion.

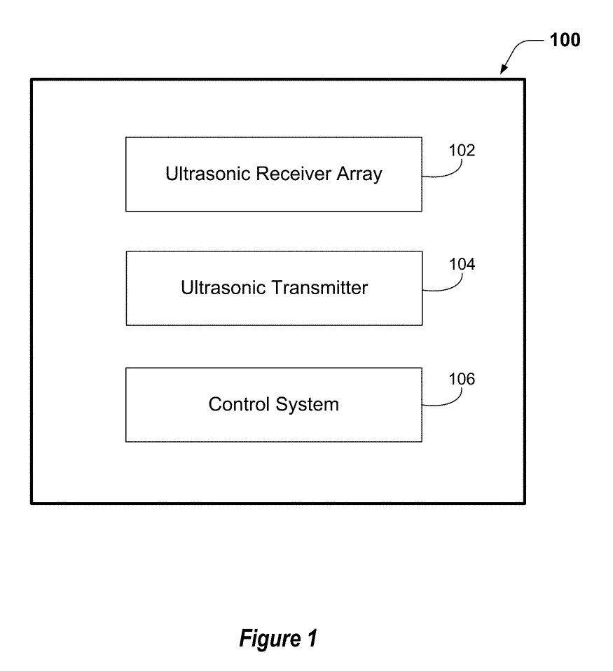

FIG. 1 is a block diagram that shows example components of an apparatus according to some implementations. In this example, apparatus 100 includes an ultrasonic receiver array 102, an ultrasonic transmitter 104 and a control system 106. Some examples of the ultrasonic receiver array 102 are descried below. According to some examples the ultrasonic transmitter 104 may be an ultrasonic plane-wave generator, such as those described in more detail below. However, in other implementations the ultrasonic transmitter 104 may include an array of ultrasonic transmitter elements, such as an array of piezoelectric micromachined ultrasonic transducers (PMUTs), an array of capacitive micromachined ultrasonic transducers (CMUTs), etc. Although shown as separate elements in FIG. 1, in some implementations the ultrasonic receiver array 102 and the ultrasonic transmitter 104 may be combined in an ultrasonic transceiver. For example, a piezoelectric receiver layer, PMUT elements in a single-layer array of PMUTs, or CMUT elements in a single-layer array of CMUTs may be used as an ultrasonic transmitter as well as an ultrasonic receiver.

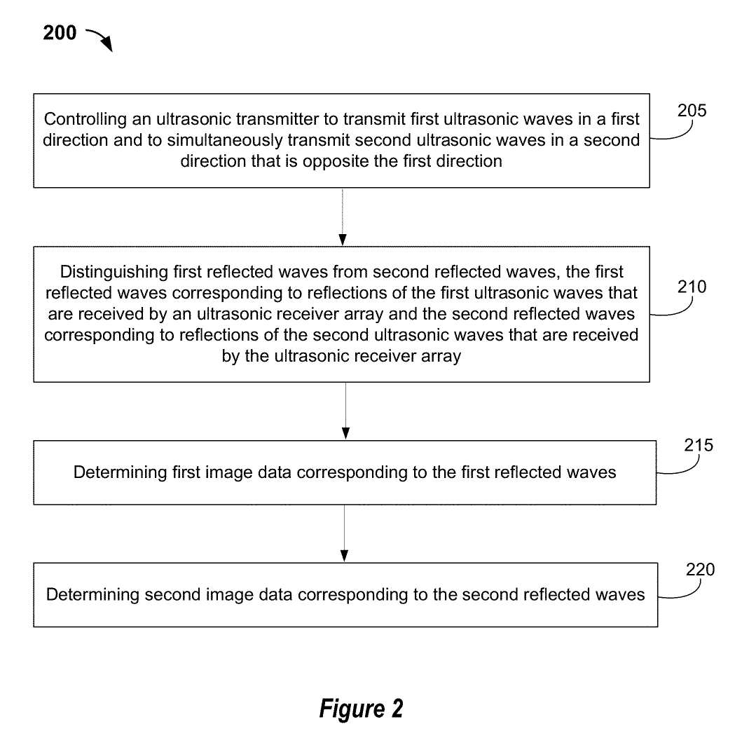

The control system 106 may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The control system 106 also may include (and/or be configured for communication with) one or more memory devices, such as one or more random access memory (RAM) devices, read-only memory (ROM) devices, etc. Accordingly, the apparatus 100 may have a memory system that includes one or more memory devices, though the memory system is not shown in FIG. 1. The control system 106 may be capable of controlling the ultrasonic transmitter 104 and of receiving and processing data from the ultrasonic receiver array 102, e.g., as described below with reference to FIG. 2 et seq.

Although not shown in FIG. 1, some implementations of the apparatus 100 may include an interface system. In some examples, the interface system may include a wireless interface system. In some implementations, the interface system may include a network interface, an interface between the control system 106 and a memory system and/or an interface between the control system 106 and an external device interface (e.g., a port or an applications processor).

The apparatus 100 may be used in a variety of different contexts, many examples of which are disclosed herein. For example, in some implementations a wearable device may include the apparatus 100. The wearable device may, for example, be a bracelet, an armband, a wristband, a ring, a headband or a patch.

FIG. 2 is a flow diagram that provides examples of biometric sensor system operations. The blocks of FIG. 2 (and those of other flow diagrams provided herein) may, for example, be performed by the apparatus 100 of FIG. 1 or by a similar apparatus. As with other methods disclosed herein, the method outlined in FIG. 2 may include more or fewer blocks than indicated. Moreover, the blocks of methods disclosed herein are not necessarily performed in the order indicated.

Here, block 205 involves controlling an ultrasonic transmitter to transmit first ultrasonic waves in a first direction and to transmit second ultrasonic waves in a second direction that is opposite the first direction. In some implementations, the control system 106 of the apparatus 100 may control the ultrasonic transmitter 104 to transmit first ultrasonic waves in a first direction and to transmit second ultrasonic waves in a second direction that is opposite the first direction. Accordingly, such implementations of the apparatus 100 are examples of what may be referred to herein as a “bidirectional ultrasonic sensor system.” In this example, block 205 involves controlling the ultrasonic transmitter to transmit the first and second ultrasonic waves simultaneously.

According to this implementation, block 210 involves distinguishing first reflected waves from second reflected waves. In this example, the first reflected waves correspond to reflections of the first ultrasonic waves that are received by an ultrasonic receiver array and the second reflected waves correspond to reflections of the second ultrasonic waves that are received by the ultrasonic receiver array. In this instance, block 215 involves determining first image data corresponding to the first reflected waves and block 220 involves determining second image data corresponding to the second reflected waves.

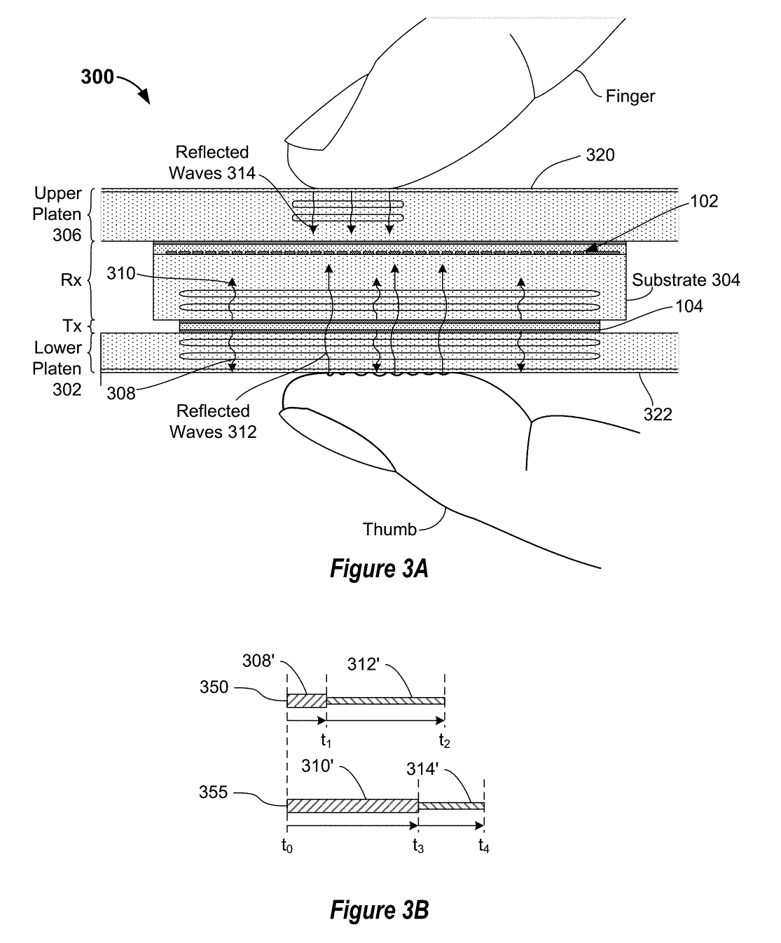

FIG. 3A shows an example of a cross-sectional view of an ultrasonic sensor system capable of performing the method of FIG. 2. The ultrasonic sensor system 300 is an example of a device that may be included in a biometric sensor system. As with other implementations shown and described herein, the types of elements, the arrangement of the elements and the dimensions of the elements illustrated in FIG. 3A are merely shown by way of example.

In this example, the ultrasonic sensor system 300 is a bidirectional ultrasonic sensor system that includes an ultrasonic receiver array 102, an ultrasonic transmitter 104 and a control system (not shown). According to this example, the ultrasonic transmitter 104 is positioned between a platen 302, which may be referred to herein as a “lower platen,” and a substrate 304 to which the ultrasonic receiver array 102 is attached. The substrate 304 may, for example, be a thin-film transistor (TFT) substrate to which circuitry for the ultrasonic receiver array 102 is attached. In this example a platen 306, which may be referred to herein as an “upper platen,” is adjacent to a stack that includes the ultrasonic receiver array 102. In some examples, the ultrasonic receiver array 102 may include an array of pixel input electrodes and sensor pixels formed in part from TFT circuitry, an overlying piezoelectric receiver layer of piezoelectric material such as PVDF or PVDF-TrFE, and an upper electrode layer positioned on the piezoelectric receiver layer sometimes referred to as a receiver bias electrode. Adhesive layers (not shown) may also be included in the sensor stack. It will be appreciated that the actual orientations of the platen 302 and the platen 306 may vary according to implementation and usage.

In some examples, the platens 302 and 306 and/or the substrate 304 may include silicon, glass, plastic, ceramic, metal, metal alloy or another such material. The thickness of the platen 302 and the platen 306 may vary according to the particular implementation. According to some implementations, the platen 302 and the platen 306 may each have a thickness that is in the range of approximately 10 microns to approximately 1000 microns, e.g., 10 microns, 20 microns, 30 microns, 40 microns, 50 microns, 100 microns, 150 microns, 200 microns, 300 microns, 400 microns, 500 microns, 600 microns, 700 microns, 800 microns, 900 microns, etc. As discussed elsewhere herein, in some implementations of the ultrasonic sensor system 300 the platen 302 and the platen 306 may have substantially different thicknesses, in order to facilitate distinguishing reflected waves 312, which are reflected from the thumb, from reflected waves 314, which are reflected from the finger. In some examples, the entire thickness of the ultrasonic sensor system 300 may be in the range of about 50 microns to about 1000 microns. In general, thinner platens and a thinner sensor stack allows the stack to be more flexible. In some implementations, the substrate 304 such as a silicon substrate or a glass TFT substrate may be thinned appreciably to increase the flexibility of the substrate and of the ultrasonic sensor system 300. For example, the glass or silicon substrates may be thinned to about 50 microns or less. In some implementations, the platen 306 may include a coating layer 320 and/or the platen 302 may include a coating layer 322. In some implementations, the coating layer 320 or coating layer 322 may serve as platen 306 or platen 302. The coating layers 320, 322 may serve as a protective layer, a smudge-resistant layer, a scratch-resistant layer, an environmentally protective layer, an acoustic impedance matching layer, an optical interference filter, or other functional layer. The coating layers 320, 322 may include a multi-layer stack of sub-layers. In some implementations, the coating layers 320, 322 may be positioned directly on the ultrasonic receiver array 102 or ultrasonic transmitter 104 and serve as a platen. In some implementations, the ultrasonic sensor system 300 may be configured without platens 302, 306 or coating layers 320, 322, with the outer surface of the ultrasonic receiver array 102 and ultrasonic transmitter 104 serving as the sensing surface. The coating layer 320 and/or the coating layer 322 may, for example, include one or more of a polycarbonate layer, a glass layer, a plastic layer such as PET, PI, PEN or PMMA, a silicone layer, an epoxy layer, an acrylic layer, or a composite layer. The coating layer 320, 322 may include a plastic or silicon-based material with a thin hard coat of diamond-like carbon (DLC), a hard coat layer, or other suitable layer. The coating layers 320, 322 or platens 302, 306 may include epoxy or acrylic-based materials with various filler materials such as aluminum oxide particles, metal or metal oxide particles, glass beads or fibers, textiles, or other particles and materials. Various silicones with embedded particles may also serve as a coating layer 320, 322 or platen 302, 306. Alternative implementations may not include the coating layer 320 or the coating layer 322.

FIG. 3A illustrates an example of block 205, in which the ultrasonic transmitter 104 is transmitting first ultrasonic waves 308 in a first direction and transmitting second ultrasonic waves 310 in a second direction that is opposite the first direction. Accordingly, the ultrasonic sensor system 300 is another example of what may be referred to herein as a “bidirectional ultrasonic sensor system.” In this example, block 205 involves controlling the ultrasonic transmitter to transmit the first and second ultrasonic waves simultaneously. The first direction, which in this example is towards a thumb of a user that is adjacent to the lower platen 302, may be referred to herein as “downward” for the sake of convenience. Likewise, the second direction, which is towards a finger of a user that is adjacent to the upper platen 306 in this example, may be referred to herein as “upward.” In this example, the first reflected waves (the reflected waves 312) are received at the ultrasonic receiver array 102 from a direction that is opposite a direction from which the second reflected waves (the reflected waves 314) are received.

In this implementation, a control system of the ultrasonic sensor system 300 is capable of distinguishing the first reflected waves, which correspond to reflections of the first ultrasonic waves that are received by the ultrasonic receiver array from a first object, from second reflected waves that correspond to reflections of the second ultrasonic waves that are received by the ultrasonic receiver array from a second object. For example, the control system may be capable of distinguishing the reflected waves 312, which are reflected from the thumb shown in FIG. 3A, from the reflected waves 314, which are reflected from the finger shown in FIG. 3A.

Accordingly, the control system may be capable of determining thumbprint image data corresponding to the reflected waves 312 and fingerprint image data corresponding to the reflected waves 314. In some implementations, a control system of the ultrasonic sensor system 300 may be capable of performing an authentication process according to the first image data, according to the second image data, or according to the first image data and the second image data. The control system may, for example, be capable of controlling the ultrasonic sensor system 300 to obtain fingerprint image data periodically and/or upon the occurrence of an event. In some such implementations, the control system may be capable of comparing currently-obtained fingerprint image data with stored fingerprint data of a patient or of another authorized person. In some examples, another device may perform part of an authentication process. The control system may be capable of receiving, via an interface system, an authentication indication from a second device indicating whether a person has been authenticated. The control system may be capable of controlling a device, allowing access to a place, allowing access to information, allowing a transaction, etc., according to the authentication indication. In some implementations, the control system may be capable of preventing or ceasing at least one function of a device if the authentication indication indicates that the user has not been authenticated.

The control system may be capable of implementing one of more of various techniques to distinguish the first reflected waves from the second reflected waves. Some such techniques involve distinguishing the first reflected waves from the second reflected waves according to different travel times from transmission of ultrasonic waves by the ultrasonic transmitter to receipt of the reflected waves by the ultrasonic receiver array. In the example shown in FIG. 3A, the upper platen 306 has been made thicker than the lower platen 302 in order to enhance this difference in travel times. In different examples, the upper platen 306 may be made thinner, thicker or substantially the same thickness as the lower platen 302.

FIG. 3B indicates the distances and times traveled by the ultrasonic waves shown in FIG. 3A, from the time of transmission by the ultrasonic transmitter 104 until the time of receipt by the ultrasonic receiver array 102. Timeline 350 indicates the distance 308′ traveled by the first ultrasonic waves 308 from launch at time t₀ to a reflection at an outer surface of lower platen 302 at time t₁, followed by a distance 312′ traveled by the reflected waves 312, which are reflected from the thumb, until a time of receipt by the ultrasonic receiver array 102 at time t₂. Timeline 355 indicates the distance 310′ traveled by the second ultrasonic waves 310 from launch at time t₀ to a reflection at an outer surface of upper platen 306 at time t₃, followed by a distance 314′ traveled by the reflected waves 314, which are reflected from the finger, until a time of receipt by the ultrasonic receiver array 102 at time t₄. It may be seen in FIG. 3B that in this example the timeline 350 is shorter than the timeline 355. Assuming that the velocity of the ultrasonic waves is approximately the same throughout the ultrasonic sensor system 300, the difference in length between the timeline 350 and the timeline 355 would indicate a corresponding difference in travel time (e.g., t₄−t₂). Decreasing the thickness of the platen 302 would tend to increase this difference in travel time. Similarly, increasing the thickness of the platen 306 would tend to increase this difference in travel time. In a similar manner, adjusting the acquisition times t₂, t₄ allows ultrasonic imaging at various depths within the thumb, finger or other biometric object positioned on either or both sides of the ultrasonic sensor system 300.

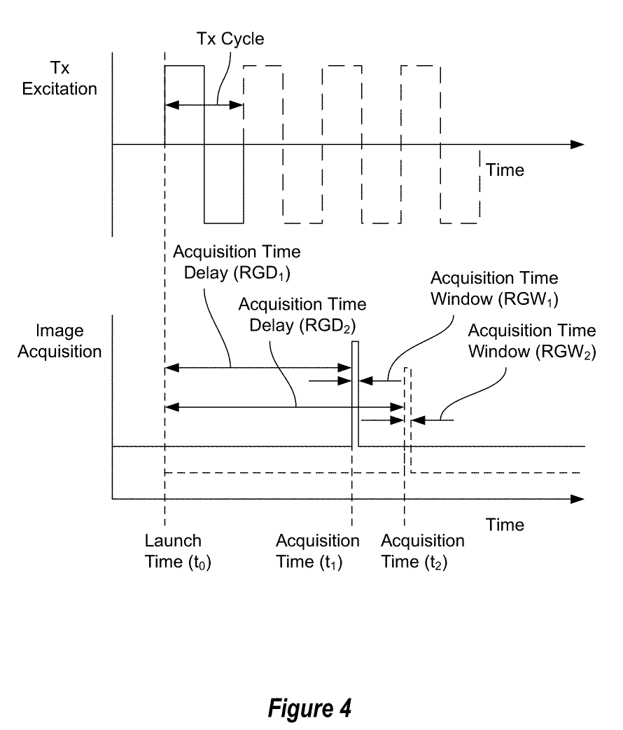

FIG. 4 includes two diagrams that illustrate an example of distinguishing first reflected waves from second reflected waves according to acquisition time delays. The upper diagram is an excitation diagram that represents the excitation voltage that is applied by a control system to an ultrasonic transmitter according to one implementation. In this example, the excitation voltage is applied via a series of square waves of alternating polarity, with one positive and one negative square wave being applied during each transmission cycle. Other implementations may involve applying waves of different shape, of different amplitude, applying a different number of waves per transmission cycle, etc.

The lower diagram of FIG. 4 shows an example of acquiring image data during two acquisition time windows, which also may be referred to as range gate windows. In this example, first image data are acquired via an ultrasonic receiver array during a first range gate window RGW₁ after a first acquisition time delay, which also may be referred to as a first range gate delay RGD₁, after the time of transmitting ultrasonic waves (shown as the “launch time” t₀ in FIG. 4.) Here, second image data are acquired via an ultrasonic receiver array during a second range gate window RGW₂ after a second acquisition time delay, which also may be referred to as a second range gate delay RGD₂, after t₀. In this example, RGW₁ begins at time t₁ and RGW₂ begins at time t₂.

The range gate delays and range gate windows may be selected to receive reflections primarily from the top or the bottom of the ultrasonic sensor system 300 shown in FIG. 3A. In this example, layer thicknesses (including the thicknesses of the platen 302 and the platen 306) of the ultrasonic sensor system 300 have been selected such that reflections received from a first side of the ultrasonic sensor system 300 will arrive before reflections from a second side of the ultrasonic sensor system 300.

Therefore, acquisition time delay RGD₁ and acquisition time window of RGW₁ may be selected such that reflected waves that are received by the ultrasonic receiver array 102 after an acquisition time delay RGD₁ and sampled during an acquisition time window of RGW₁ will generally be reflected from a portion of a first object proximate to or positioned upon the first side of the ultrasonic sensor system 300, which will be from the thumb adjacent to the platen 302 in the example of FIG. 3A. Accordingly, acquisition time delay RGD₁ and acquisition time window of RGW₁ may be selected such that at least some of the reflected waves that are received by the ultrasonic receiver array 102 after RGD₁ and sampled during RGW₁ will correspond with timeline 350 of FIG. 3B, which indicates the total distance and time traveled by the first ultrasonic waves 308 and the reflected waves 312.

Likewise, acquisition time delay RGD₂ and acquisition time window of RGW₂ may be selected such that reflected waves that are received by the ultrasonic receiver array after an acquisition time delay RGD₂ and sampled during an acquisition time window of RGW₂ will generally be reflected from a portion of a second object proximate to or positioned upon the second side of the ultrasonic sensor system 300, which will be from the finger adjacent to the platen 306 in the example of FIG. 3A. Accordingly, acquisition time delay RGD₂ and acquisition time window of RGW₂ may be selected such that at least some of the reflected waves that are received by the ultrasonic receiver array 102 after RGD₂ and sampled during RGW₂ will correspond with timeline 355 of FIG. 3B, which indicates the total distance and time traveled by the second ultrasonic waves 310 and the reflected waves 314.

Accordingly, acquisition time delays and acquisition time windows may be selected for distinguishing first reflected waves from second reflected waves in some implementations of block 210 of FIG. 2. However, other implementations may involve other methods of distinguishing the first reflected waves from the second reflected waves. Some such implementations may make the distinction based, at least in part, on frequency-dependent content of the first reflected waves and the second reflected waves. In some such implementations, the frequency-dependent content of the transmitted first ultrasonic waves may differ from that of the transmitted second ultrasonic waves. Alternatively, or additionally, one or more layers and/or ultrasonic transmitter excitation signals of the ultrasonic sensor system 300 may be selected to cause differing frequency-dependent content of the reflected waves, e.g., by absorbing or delaying certain frequency ranges, by using a frequency-dependent delay layer, etc. For example, a frequency-dependent delay layer, which may be used as one or other of the platens 302, 306 or otherwise coupled thereto, has a characteristic speed of sound that may be faster or slower with changes in frequency of the launched ultrasonic wave. One pass of a sound wave through the delay layer at a first ultrasonic frequency results in a differential time delay when a different ultrasonic frequency is used. Two passes through the delay layer results in twice the differential time delay as one pass. A biometric object such as a finger or thumb positioned on one side of the ultrasonic sensor system 300 may be selectively imaged at a time t₁ using launched ultrasonic waves of a first frequency f₁, then a second biometric object positioned on the opposite side of the ultrasonic sensor system 300 may be selectively imaged at a time t₂ using launched waves of a second frequency f₂, with f₁ and t₁ selected so that an object on a first side is imaged and an object on the second side is not imaged, and with f₂ and t₂ selected so that an object on the first side is not imaged and an object on the second side is imaged. In another example, an acoustic cavity placed in or coupled to one or the other of the platens 302, 306 may be configured to pass some ultrasonic frequencies and absorb or reflect other ultrasonic frequencies, so that an object positioned on a first side of the ultrasonic sensor system 300 may be imaged using a first ultrasonic frequency f₁ that is passed by the acoustic cavity, whereas a second ultrasonic frequency f₂ that is not passed by the acoustic cavity may be used to image an object positioned on a second side of the ultrasonic sensor system 300.

Some implementations may involve distinguishing the first reflected waves from the second reflected waves according to one or more image processing methods. For example, some such implementations may involve using software to subtract one image from another, known image. For example, referring to FIG. 3A, thumbprint image data obtained from the thumb adjacent to the platen 302 of the ultrasonic sensor system 300 may be subtracted from fingerprint image data obtained from the finger adjacent to the platen 306, in order to reduce image artifacts from one side to the other.

Some implementations may involve distinguishing the first reflected waves from the second reflected waves based, at least in part, on temperature data received from one or more temperature sensors of a biometric sensor system that includes the ultrasonic sensor system 300. For example, if the biometric sensor system is deployed in a wearable device, one portion of the biometric sensor system (e.g., one platen) may be in continuous contact with a corresponding portion of a user's body (such as a wrist, a finger, etc.) and may therefore tend to be warmer. An opposing side of the biometric sensor system may occasionally be touched by a user's finger, but may otherwise not be in contact with the user's body. This side may tend to be relatively cooler until touched. In some implementations, a temperature sensor positioned near or on one or the other of the imageable surfaces of the biometric sensor system may indicate a rise or fall in temperature as a finger or other warm object comes in contact with the sensor surface or is removed from contact with the sensor system.

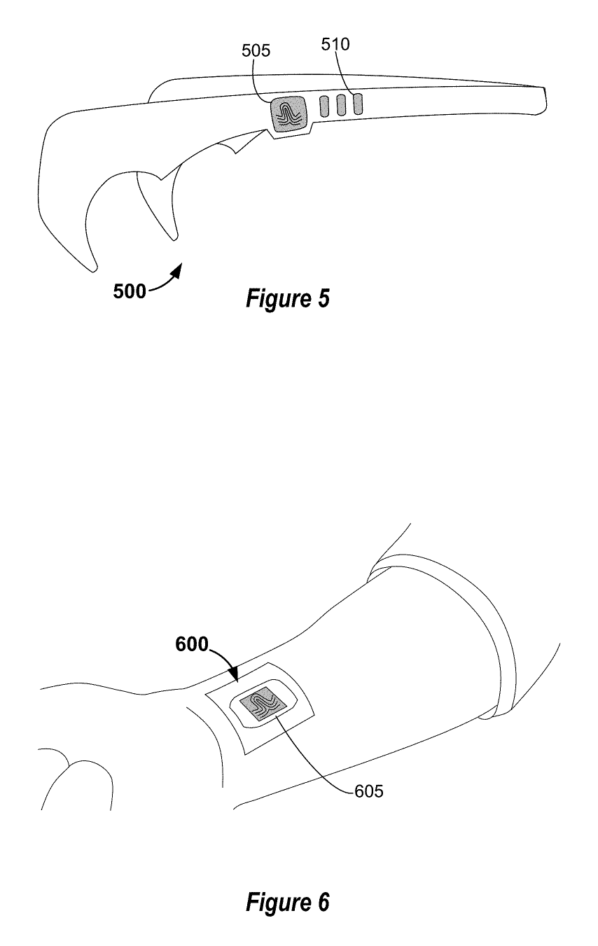

Accordingly, in some implementations a wearable device may include a biometric sensor system. In some such implementations, the biometric sensor system may include a bidirectional ultrasonic sensor system, such as the ultrasonic sensor system 300. The wearable device may, for example, be a bracelet, an armband, a wristband, a ring, a headband or a patch. In some implementations, the entire thickness of the biometric sensor system may be quite small, e.g., in the range of 50 microns to 500 microns. In some implementations, the biometric sensor system may be flexible enough for use in a flexible wearable device. Some examples of wearable device implementations will now be described with reference to FIG. 5 et seq.

FIG. 5 shows an example of a headband that includes a biometric sensor system. Here, the headband 500 includes a biometric sensor system 505. In some implementations, the biometric sensor system 505 may include a bidirectional ultrasonic sensor system, such as the ultrasonic sensor system 300. In this example, the biometric sensor system 505 is capable of obtaining first image data, such as fingerprint image data, from an outer surface of the biometric sensor system 505. In some implementations, the biometric sensor system 505 may be capable of obtaining second image data from an inner surface of the biometric sensor system 505, or from an inner surface of the headband 500 that is adjacent the biometric sensor system 505. The second image data may correspond to tissue images, follicle images, etc., according to the particular implementation. According to some examples, the headband 500 may include an acoustic matching layer, such as a gel (not shown), on the inner surface of the headband 500 that is adjacent the biometric sensor system 505. In some implementations, a control system of the biometric sensor system 505 may be capable of performing an authentication process according to the first image data, according to the second image data, or according to the first image data and the second image data. In some implementations, the headband 500 may include a wired or wireless interface that allows the biometric sensor system 505 to be capable of wireless communication with another device. In some implementations, the headband 500 may include one or more buttons 510. Buttons 510 may allow, for example, electronic portions of the headband and the biometric sensor system 505 to be turned on or off, a volume to be adjusted, a track to be played, an application to be located and/or selected, or another function to be performed.

FIG. 6 shows an example of a patch that includes a biometric sensor system. In this implementation, the patch 600 includes a biometric sensor system 605. In some implementations, the biometric sensor system 605 may include a bidirectional ultrasonic sensor system, such as the ultrasonic sensor system 300. In this example, the biometric sensor system 605 is capable of obtaining first image data, such as fingerprint image data, from an outer surface of the biometric sensor system 605. In some implementations, the biometric sensor system 605 may be capable of obtaining second image data from an inner surface of the biometric sensor system 605, or from an inner surface of the patch 600 that is proximate the biometric sensor system 605. The second image data may correspond to tissue images such as skin tissue images, bone tissue images, blood vessel images, follicle images, etc., depending on the particular implementation. According to some examples, the patch 600 may include an acoustic matching layer, such as a gel, on the inner surface of the patch 600. In some implementations, a control system of the biometric sensor system 605 may be capable of performing an authentication process according to the first image data, according to the second image data, or according to the first image data and the second image data. In some implementations, the patch 600 may include a wireless interface that allows the biometric sensor system 605 to be capable of wireless communication with another device.

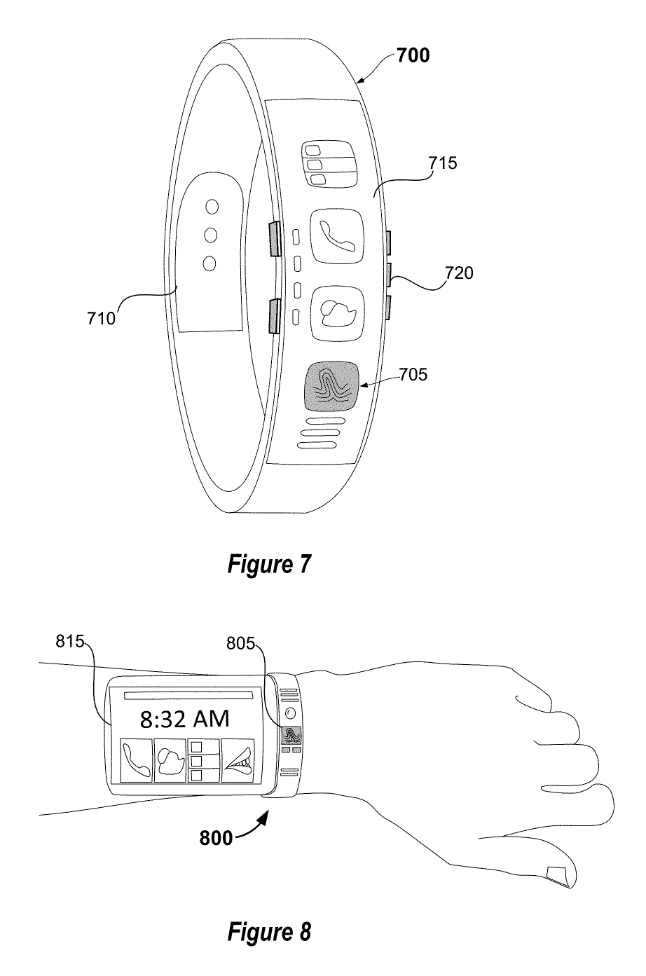

FIG. 7 shows an example of a wristband that includes a biometric sensor system. Here, the wristband 700 includes a biometric sensor system 705. In some implementations, the biometric sensor system 705 may include a bidirectional ultrasonic sensor system, such as the ultrasonic sensor system 300. In this example, the biometric sensor system 705 is capable of obtaining first image data, such as fingerprint image data, from an outer surface of the biometric sensor system 705.

In some implementations, the biometric sensor system 705 may be capable of obtaining second image data from an inner surface of the biometric sensor system 705, or from an inner surface of the wristband 700 that is adjacent to at least a portion of the biometric sensor system 705. The portion of the biometric sensor system 705 that is capable of acquiring the first image data may or may not be adjacent to the portion of the biometric sensor system 705 that is capable of acquiring the second image data, depending on the particular implementation. The implementation of the biometric sensor system 705 shown in FIG. 7, for example, includes an inner portion 710 that is capable of obtaining the second image data. The second image data may correspond to tissue images, follicle images, etc., according to the particular implementation. According to some examples, the wristband 700 may include an acoustic matching layer, such as a gel, on the inner surface of the wristband 700 that is adjacent to at least a portion the biometric sensor system 705 (e.g., on the inner portion 710).

According to some implementations, a control system of the biometric sensor system 705 may be capable of performing an authentication process according to the first image data, according to the second image data, or according to the first image data and the second image data. In some implementations, the wristband 700 may include a wireless interface that allows the biometric sensor system 705 to be capable of wireless communication with another device.

In this example, the wristband 700 includes a flexible display 715, which is shown displaying various icons. In some implementations of the wristband 700, a touch sensor system may overlay at least part of the display 715. According to some such implementations, a user may interact with the displayed icons in order to control functionality of the wristband 700, such as selecting from a menu, making a telephone call, querying a weather application, etc. In this example, a user input system of the wristband 700 also includes various buttons 720.

FIG. 8 shows an example of an armband that includes a biometric sensor system. Here, the armband 800 includes a biometric sensor system 805. In some implementations, the biometric sensor system 805 may include a bidirectional ultrasonic sensor system, such as the ultrasonic sensor system 300. In this example, the biometric sensor system 805 is capable of obtaining first image data, such as fingerprint image data, from an outer surface of the biometric sensor system 805.

In some implementations, the biometric sensor system 805 may be capable of obtaining second image data from an inner surface of the biometric sensor system 805, or from an inner surface of the armband 800 that is adjacent to at least a portion of the biometric sensor system 805. The portion of the biometric sensor system 805 that is capable of acquiring the first image data may or may not be adjacent to the portion of the biometric sensor system 805 that is capable of acquiring the second image data, depending on the particular implementation. The second image data may correspond to tissue images, follicle images, etc., according to the particular implementation. According to some examples, the armband 800 may include an acoustic matching layer, such as a gel, on the inner surface of the armband 800 that is adjacent to at least a portion the biometric sensor system 805.

In some implementations, a control system of the biometric sensor system 805 may be capable of performing an authentication process according to the first image data, according to the second image data, or according to the first image data and the second image data. In some implementations, the armband 800 may include a wireless interface that allows the biometric sensor system 805 to be capable of wireless communication with another device.

In this example, the armband 800 includes a flexible display 815, which is shown displaying various icons. In some implementations of the armband 800, a touch sensor system may overlay at least part of the display 815. According to some such implementations, a user may interact with the displayed icons in order to control functionality of the armband 800, such as making a telephone call, querying a weather application, selecting a menu item, controlling a playlist, etc.

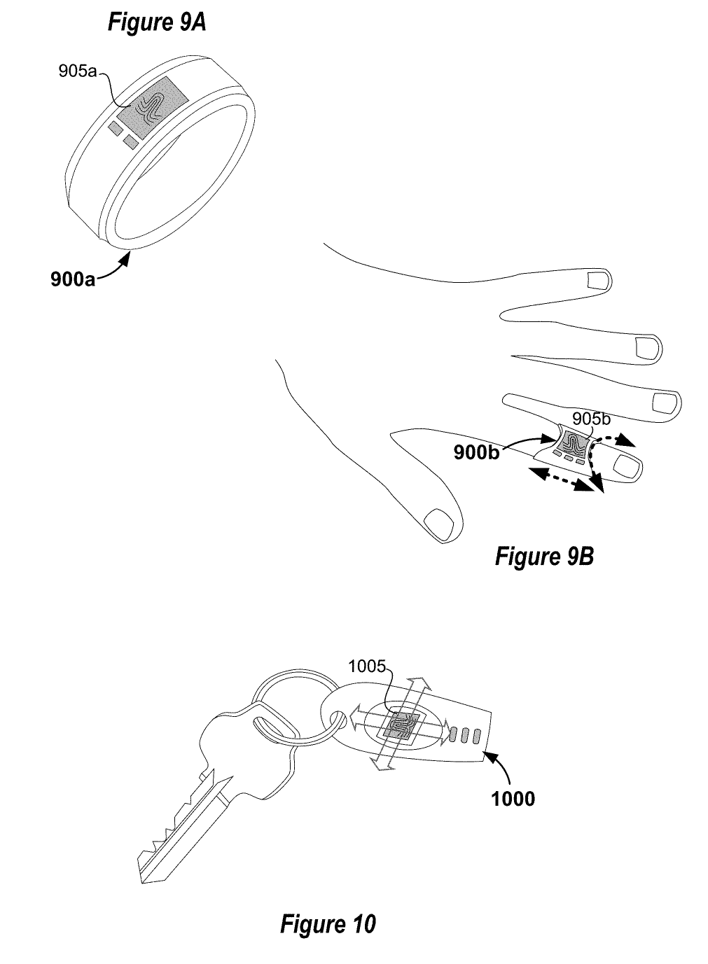

FIGS. 9A and 9B show examples of rings that include biometric sensor systems. In this implementation, the ring 900a includes a biometric sensor system 905a and the ring 900b includes a biometric sensor system 905b. In some implementations, the biometric sensor system 905a and/or the biometric sensor system 905b may include a bidirectional ultrasonic sensor system, such as the ultrasonic sensor system 300. In these examples, the biometric sensor systems 905a and 905b are both capable of obtaining first image data, such as fingerprint image data, from an outer surface of the biometric sensor systems 905a and 905b. In some implementations, the biometric sensor system 905a may be capable of obtaining second image data from an inner surface of the biometric sensor system 905a, or from an inner surface of the ring 900a that is proximate the biometric sensor system 905a. Similarly, in some implementations the biometric sensor system 905b may be capable of obtaining second image data from an inner surface of the biometric sensor system 905b, or from an inner surface of the ring 900b that is proximate the biometric sensor system 905b. The second image data may correspond to tissue images such as skin tissue images, bone tissue images, blood vessel images, follicle images, etc., depending on the particular implementation. According to some examples, the ring 900a or the ring 900b may include an acoustic matching layer, such as a gel, on an inner surface. In some implementations, a control system of the biometric sensor system 905a, the biometric sensor system 905b, or both, may be capable of performing an authentication process according to the first image data, according to the second image data, or according to the first image data and the second image data. In some implementations, the ring 900a, the ring 900b, or both the ring 900a and the ring 900b may include a wireless interface that allows wireless communication with another device. In some implementations, the ring 900a or 900b may be twisted or slid along a finger and the sliding or twisting motion detected by the biometric sensor system 905a or 905b. A function such as pointing and remotely activating a wall switch, operating a volume control on a remote television screen, or interacting with an application may be performed in response to the detected motion. The functions accessible to the ring 900a or 900b may be adjusted depending on whether a user wearing the ring 900a or 900b is authorized and/or authenticated. Similarly, a finger from an opposite hand may be twisted or slid along an outer surface of ring 900a or 900b and the sliding or twisting motion detected by the biometric sensor system 905a or 905b to initiate, access or otherwise perform a function. In some implementations, the biometric sensor system 905a or 905b may authenticate a user based on images obtained from a finger or other biometric object placed on the outer surface of the biometric sensor system 905a or 905b, and adjust accessible functions accordingly.

FIG. 10 shows an example of a key fob that includes a biometric sensor system. In this implementation, the key fob 1000 includes a biometric sensor system 1005. In some implementations, the biometric sensor system 1005 may include a bidirectional ultrasonic sensor system, such as the ultrasonic sensor system 300. However, in some implementations the biometric sensor system 1005 may not be a bidirectional ultrasonic sensor system. In this example, the biometric sensor system 1005 is capable of obtaining at least first image data, such as fingerprint image data or thumbprint image data, from an outer surface of the biometric sensor system 1005. In some implementations, the biometric sensor system 1005 may be capable of obtaining second image data from an inner surface of the biometric sensor system 1005, or from an inner surface of the key fob 1000 that is proximate the biometric sensor system 1005. The second image data may correspond to tissue images such as skin tissue images, bone tissue images, blood vessel images, follicle images, etc., depending on the particular implementation. In some implementations, a control system of the biometric sensor system 1005 may be capable of performing an authentication process according to the first image data, according to the second image data, or according to the first image data and the second image data. In some implementations, the key fob 1000 may include a wireless interface that allows the biometric sensor system 1005 to be capable of wireless communication with another device, such as a door of a vehicle or house. Movements or taps of a finger placed on the biometric sensor system 1005 may be detected and functions performed accordingly. In some implementations, the biometric sensor system 1005 may authenticate a user based on images obtained from a finger placed on the outer surface of the biometric sensor system 1005 and adjust functions accordingly.

In some implementations, a control system of a biometric sensor system (such as the biometric sensor systems described above with reference to FIGS. 5-10) may be capable of controlling an ultrasonic sensor system to obtain fingerprint or thumbprint image data periodically and/or upon the occurrence of an event. In some such implementations, the control system may be capable of comparing currently-obtained fingerprint image data or thumbprint image data with stored fingerprint or thumbprint data of a patient or of another authorized person. In some examples, another device may perform part of an authentication process. The control system may be capable of receiving, via an interface system, an authentication indication from a second device indicating whether a person has been authenticated. The control system may be capable of controlling a device, allowing access to a place, allowing access to information, etc., according to the authentication indication. In some implementations, the control system may be capable of preventing or ceasing at least one function of a device if the authentication indication indicates that the user has not been authenticated.

Some wearable devices may be capable of validating a wearer on a continuous or nearly continuous basis. According to some such examples, the control system may be capable of obtaining a tissue image, a bone image, or both a tissue image and a bone image, from a first side of the wearable device. For example, images received from an inside portion of a ring, patch, armband, wristband, headband, etc., may be acquired and evaluated to ensure that a device remains on a particular individual. Alternatively, or additionally, some wearable devices may be capable of providing authentication from time to time (e.g., to make a purchase, to provide access to confidential information, to provide access to another device or a controlled area, to time stamp an event, to deliver a drug, etc.). According to some such implementations, a control system may be capable of obtaining a fingerprint image from a second side of the wearable device. In some examples, a control system may be capable of performing an authentication process based, at least in part, on images obtained from the first side or the second side of the wearable device. In some implementations such as those shown in FIGS. 5-10, an outward-facing portion of a wearable device may be capable of providing occasional user authentication or re-authentication by obtaining fingerprint image data or thumbprint image data from a user.

As noted above, some wearable device implementations may be capable of gesture detection, e.g., by determining that a wearable device has been moved relative to a corresponding part of a user's body. For example, in some implementations a control system may be capable of determining whether the ring 905b shown in FIG. 9B has been rotated or translated along a user's finger. In some implementations, a control system may be capable of determining whether a user's finger or thumb has moved relative to the biometric sensor system 1005 of the key fob 1000. In some implementations, gestures may be detected on one side of the biometric sensor while the other side remains in contact with a user to provide continuous authentication. In some such implementations, detected gestures may be forms of user input. Different types of gestures may correspond with different functionality desired by a user. Such functionality may involve control of another device with which the biometric sensor system is capable of communicating, e.g., via wireless communication.

Accordingly, in some implementations the control system may be capable of detecting a motion of the biometric sensor system, or of detecting a motion relative to the biometric sensor system, and of producing a motion-detection signal that corresponds with a detected motion. The control system may, for example, be capable of detecting motion according to changes in at least one of the first image data or the second image data described above. The control system may be capable of producing a motion-detection signal that corresponds with a detected motion. If the particular implementation includes a wireless interface, the control system may be capable of transmitting the motion-detection signal via the wireless interface.

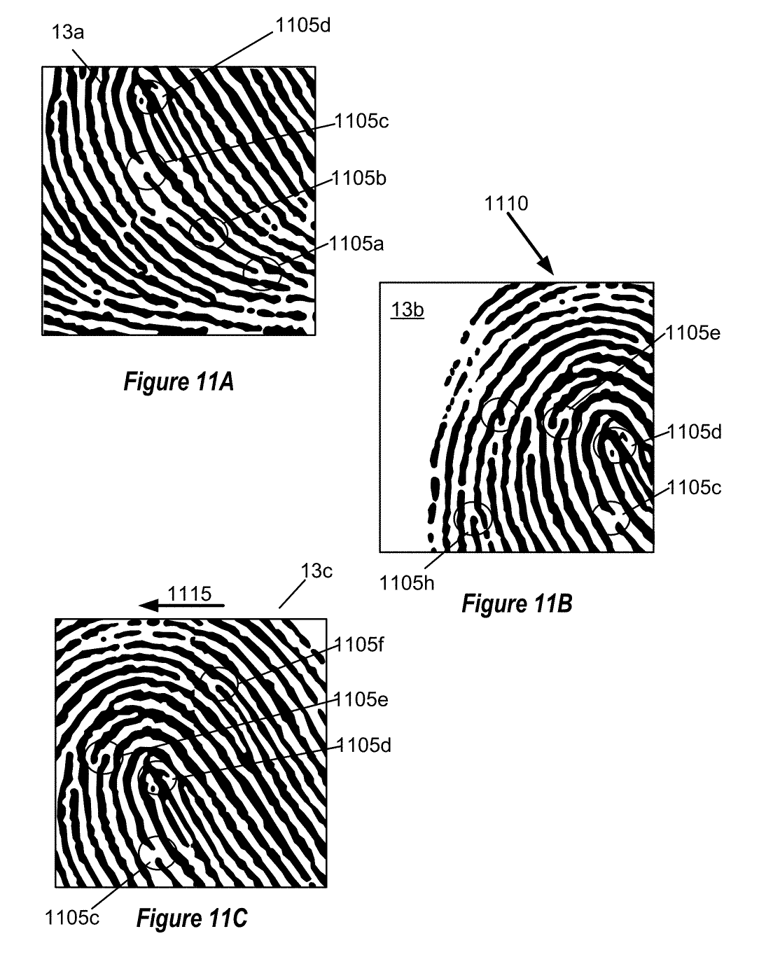

FIGS. 11A-11C provide examples of detecting motion according to changes in fingerprint image data or thumbprint image data. FIGS. 11A-11C show examples of partial fingerprint images and fingerprint features. In this example, FIGS. 11A-11C correspond to partial fingerprint images 13a-13c that have been obtained from a relatively small fingerprint sensor with an approximately square active area at three different times. According to some implementations, a subset of fingerprint features corresponding to each of the partial fingerprint images 13a-c may be extracted from each corresponding subset of fingerprint image information and a partial fingerprint template may be generated. The partial fingerprint template may correspond with features shown in one or more of the partial fingerprint images 13a-c. The partial fingerprint template may, for example, include the types, locations and/or spacing of the fingerprint minutiae 1105a-1105d shown in FIG. 11A, of the fingerprint minutiae 1105c-1105e and 1105h shown in FIG. 11B and of the fingerprint minutiae 1105c-1105f shown in FIG. 11C. In some implementations, one or more centroids may be determined. Each centroid may correspond to multiple fingerprint minutiae or keypoints.

By comparing fingerprint features that are determined at a first time, at which the partial fingerprint image 13a was obtained, with fingerprint features that are determined at a second time, at which the partial fingerprint image 13b was obtained, the relative motion of the corresponding finger relative to the biometric sensor system may be determined. The direction of relative motion is shown by the arrow 1110 in FIG. 11B. In some implementations, a control system may be capable of determining the direction of relative motion by comparing individual fingerprint features at the first and second times, such as the fingerprint minutiae 1105c and 1105d that may be seen in FIGS. 11A and 11B.

Similarly, by comparing fingerprint features that are determined at the second time, at which the partial fingerprint image 13b was obtained, with fingerprint features that are determined at a third time, at which the partial fingerprint image 13c was obtained, another relative motion of the corresponding finger relative to the biometric sensor system may be determined. The direction of relative motion is shown by the arrow 1115 in FIG. 11C. In some implementations, a control system may be capable of determining the direction of relative motion by comparing individual fingerprint features at the first and second times, such as the fingerprint minutiae 1105c, 1105d and 1105e that may be seen in FIGS. 11B and 11C.

In alternative implementations, a control system may be capable of determining the direction of relative motion by comparing other individual fingerprint features at different times, such as one or more ridge patterns, valley patterns, whorls, bifurcations, etc. In other implementations, a control system may be capable of determining the direction of relative motion by comparing the position of a centroid that corresponds to multiple fingerprint features at different times. In some implementations, the control system may be capable of determining a rotation or a rate of rotation based on a rotation of the fingerprint features at different times. The control system may produce a motion-detection signal that corresponds with the detected motion, such as a translation or a rotation, or a rate of translation or a rate of rotation. In some implementations, the control system may recognize one or more touches or taps of a finger, and generate a motion-detection signal that corresponds with the detected number of taps or rate of tapping.

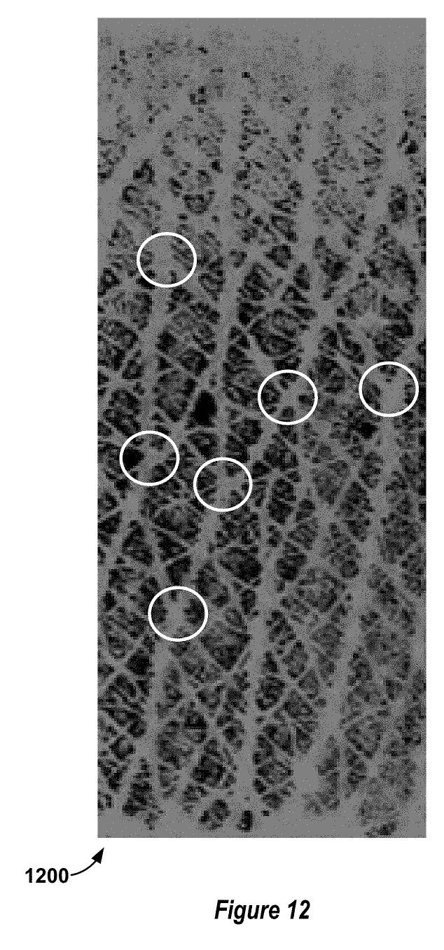

FIG. 12 shows examples of wrist image features. The image 1200 of FIG. 12 is an actual image of a user's wrist that is based on data received from an ultrasonic sensor system. Accordingly, features such as those shown in the image 1200 are like those that could be obtained from an inside portion of the patch 600 shown in FIG. 6, the wristband 700 shown in FIG. 7, the armband 800 of FIG. 8, etc.

The image 1200 indicates several types of distinctive features that may be identified and used for detecting relative motion. Several distinctive intersections of curvilinear features and relatively straight linear features are shown within circles in FIG. 12. According to some implementations, a control system may be capable of determining the direction of relative motion by comparing these individual intersections or other individual features at different times, such as one or more curvilinear features, one or more relatively straight linear features, etc. In other implementations, a control system may be capable of determining the direction of relative motion by comparing the position of a centroid that corresponds to multiple features at different times, such as a centroid that corresponds to multiple intersections of the type shown in the image 1200. In some implementations, the distinctive features of a wrist or other body part may be stored and used later for authentication of a user or to verify that the biometric sensor system had not been removed from a particular location on a body where the sensor system was initially placed.

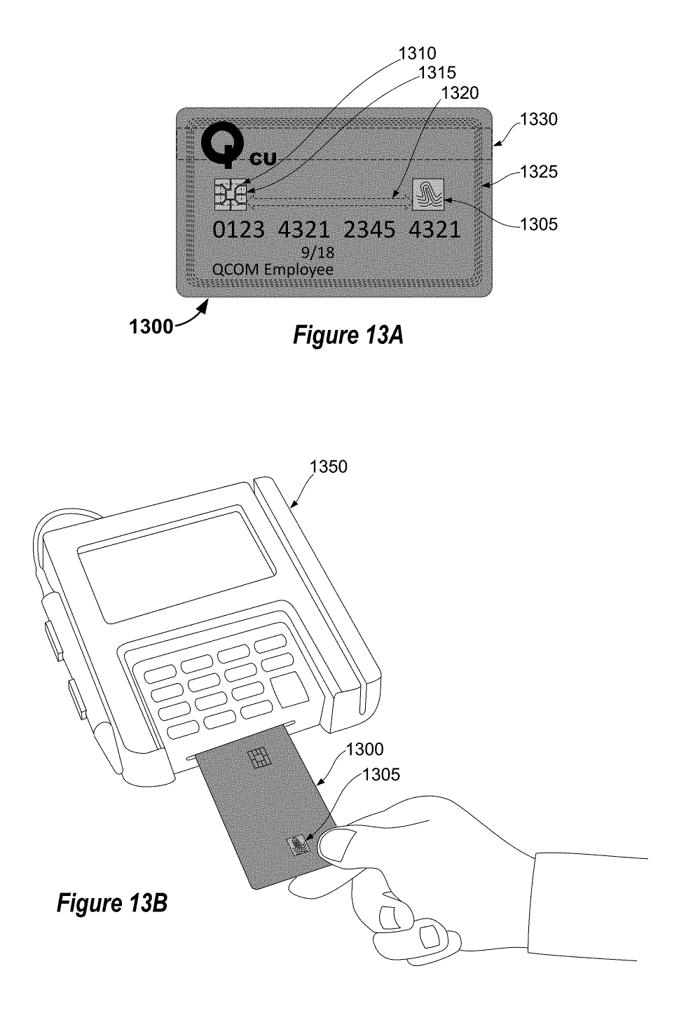

FIG. 13A shows an example of a smart card that includes a biometric sensor system. In this implementation, the smart card 1300 includes a biometric sensor system 1305. In some implementations, the biometric sensor system 1305 may include a bidirectional ultrasonic sensor system, such as the ultrasonic sensor system 300. However, in some implementations the biometric sensor system 1305 may not be a bidirectional ultrasonic sensor system.

In this example, the smart card 1300 includes an ultrasonic receiver array, an ultrasonic transmitter and a control system. The ultrasonic receiver array and the ultrasonic transmitter may be like those described elsewhere herein, e.g., with reference to FIGS. 1-3. In this example, the control system is capable of controlling the ultrasonic transmitter to transmit first ultrasonic waves in a first direction and to simultaneously transmit second ultrasonic waves in a second direction that is opposite the first direction. The first direction may be towards a first side of the smart card and the second direction may be towards a second side of the smart card.

In this implementation, the control system is capable of distinguishing first reflected waves from second reflected waves. Here, the first reflected waves correspond to reflections of the first ultrasonic waves that are received by the ultrasonic receiver array and the second reflected waves corresponding to reflections of the second ultrasonic waves that are received by the ultrasonic receiver array. In this example, the control system is capable of determining first image data corresponding to the first reflected waves and determining second image data corresponding to the second reflected waves.

Accordingly, the control system is capable of obtaining at least first image data from a first surface of the biometric sensor system 1305. For example, the first surface may correspond with the top surface of the smart card, which is shown in FIG. 13A. The first image data may include fingerprint image data, thumbprint image data, or another type of data, depending on the particular implementation.

In some implementations, the biometric sensor system 1305 may be capable of obtaining second image data from a second surface of the biometric sensor system 1305, or from a second surface of the smart card 1300 that is proximate the biometric sensor system 1305. The second image data may include fingerprint image data, thumbprint image data, or another type of data, depending on the particular implementation. In some implementations, a control system of the smart card 1300 may be capable of performing an authentication process according to the first image data, according to the second image data, or according to the first image data and the second image data.

FIG. 13B shows an example of using the smart card of FIG. 13A. In this example, the smart card 1300 is capable of “pinch authentication” based on images of a user's thumb on one side of the biometric sensor system and a user's finger on another side of the biometric sensor system. Accordingly, in this example a control system of the smart card 1300 is capable of performing an authentication process according to first image data and second image data, wherein the first image data or the second image data include fingerprint image data and the first image data or the second image data include image data corresponding to a thumb.

In addition to providing bidirectional authentication functionality, in this example the control system of the smart card 1300 includes a chip 1310 that is capable of communication with a smart card reader 1350. In this example, contact pads 1315 and embedded electrical traces 1320 may provide electrical connectivity between the chip 1310 and the biometric sensor system 1305. This implementation of the smart card 1300 also includes a magnetic stripe 1330, which allows the smart card 1300 to be capable of communication with legacy smart card readers that are not configured for communication via the chip 1310. In this example, the smart card 1300 includes a radio frequency antenna 1325 that allows the biometric sensor system 1305 to be capable of wireless communication with another device.

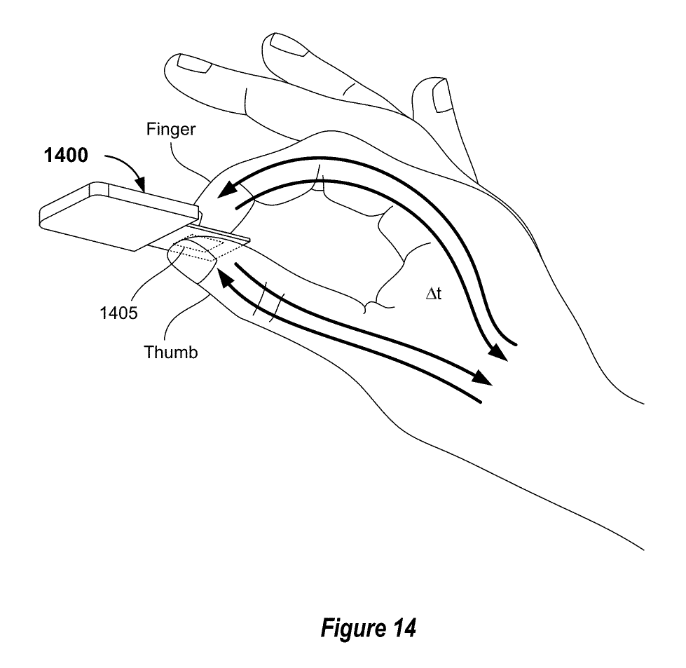

FIG. 14 shows an example of pulse and/or liveness detection with a biometric sensor system. In this implementation, the biometric sensor system 1400 includes an ultrasonic sensor system 1405. In some implementations, the ultrasonic sensor system 1405 may include a bidirectional ultrasonic sensor system, such as the ultrasonic sensor system 300 and a control system as described, for example, with reference to FIGS. 1-3. In this example, the control system is capable of controlling the ultrasonic sensor system 1405 to transmit first ultrasonic waves in a first direction and to simultaneously transmit second ultrasonic waves in a second direction that is opposite the first direction. The control system may be capable of distinguishing first reflected waves from second reflected waves. The first reflected waves may correspond to reflections of the ultrasonic waves that are transmitted in the first direction and the second reflected waves may correspond to reflections of the ultrasonic waves that are transmitted in the second direction. In this example, the control system may be capable of determining first image data corresponding to the first reflected waves and determining second image data corresponding to the second reflected waves. Accordingly, the control system may be capable of obtaining image data from at least a first surface of the ultrasonic sensor system 1405 and image data from at least a second surface of the ultrasonic sensor system 1405. For example, the first surface may correspond with a top surface of the biometric sensor system 1400 and the second surface may correspond with a bottom surface of the biometric sensor system 1400. In some implementations, the control system may be capable of performing an authentication process, a pulse rate (or heartrate) detection process, and/or a liveness determination process according to the first image data, the second image data, or both the first image data and second image data. In some implementations, the liveness determination process may include determining whether a biometric object exhibits a pulse or a pulse rate.

In example shown in FIG. 14, the biometric sensor system may be capable of pinch authentication based on images of a user's thumb on one side of the biometric sensor system and a user's finger on another side of the biometric sensor system. Accordingly, the control system may be capable of performing an authentication process, a liveness determination process and/or a pulse rate detection process according to the first image data and second image data. In some implementations, the first image data or the second image data may include fingerprint image data corresponding to a finger and the first image data or the second image data may include fingerprint image data corresponding to a thumb. In addition to providing bidirectional authentication, liveness determination and/or pulse rate detection functionality, the control system may be capable of wired or wireless communication with, for example, a network device, a storage device or an external electronic device.

In the example shown in FIG. 14, biometric sensor system 1400 may be capable of one- or two-sided imaging, authentication, liveness determination and/or pulse or pulse rate detection along with other functions. A liveness determination may be made, for example, by imaging a finger and a thumb or other portion of a body positioned against one or more surfaces of the biometric sensor system 1400 and detecting pulsing of blood (e.g. liveness) over time via a phase difference caused by different arterial path lengths within the body portions. Detection of a pulse, a pulse rate or a phase difference may be used alone or with other factors such as a finger/non-finger determination process to make a liveness determination of a biometric object positioned against one or both sides of the biometric sensor system 1400. The ultrasonic sensor system 1405 may be used to image fingerprints and/or acquire planar scans of the biometric object at various depths to obtain three-dimensional images of underlying tissue. The ultrasonic sensor system 1405 may send ultrasonic waves for imaging in two directions away from the sensor. With proper selection of range-gate timing, ultrasonic images may be obtained of a biometric object through platens above and below the ultrasonic sensor system 1405. The low profile of the ultrasonic sensor system 1405 may allow imaging of underlying tissue on a continuous or quasi-continuous basis and to authenticate, validate, determine liveness and/or detect a heartrate of a user when desired. In other configurations, one finger may be placed on an outer surface of a two-sided ultrasonic sensor system 1405 configured in a headband, armband, wristband, ring, skin patch or other body-conforming device, where the inner surface of the ultrasonic sensor system is in contact with the body part to provide arterial path-length differences and to allow determination of liveness and/or other arterial characteristics.

In some implementations, a bidirectional ultrasonic sensor system may be used to measure various characteristics of arteries at two different positions at substantially the same time, e.g., a pulse pressure waveform or an indication of volumetric flow of blood, as two different fingers touching opposite sides of the bidirectional sensor system may present two different locations in the arterial tree within a body. For example, two fingers of the same hand may be in contact with the double-sided sensor or two fingers from two hands of a user may contact the double-sided sensor at the same time, where characteristics of arteries in the fingers may be measured. In some implementations, one side of a bidirectional sensor system may be integrated into a skin patch that is adhered to a portion of a body such as an arm of a user. A finger of the user may be pressed against the outer side of the double-sided sensor. The biometric sensor system may measure characteristics of the radial artery in the arm and the digital artery in the finger at substantially the same time. In some implementations, phase differences between pressure pulses in the arm and the finger due to the beating of a heart may be detected and a pulse, a pulse rate or an arterial characteristic may be determined from the phase difference. In some implementations, a pulse and/or a pulse rate may be determined by pressing a finger and a thumb against each side of a bidirectional ultrasonic sensor as shown in FIG. 14, acquiring two or more sets of image data from each side of the sensor, applying a smoothing filter, and detecting out-of-phase throbbings of the finger and thumb based on changing ridge-to-valley areas as pressure pulses from a beating heart change the amount of contact area between ridges of the finger/thumb and the sensor. The time difference (delta t) represents a time delay between the peak pulse pressure at the thumb and the peak pulse pressure at the finger, as the arterial path length is generally different between a thumb and a finger. A non-zero value of the time difference may aid in affirming liveness of the finger and the thumb. Measuring the time difference between consecutive pressure peaks at either the thumb or the finger may allow determination of a pulse rate by taking the reciprocal of the time difference.

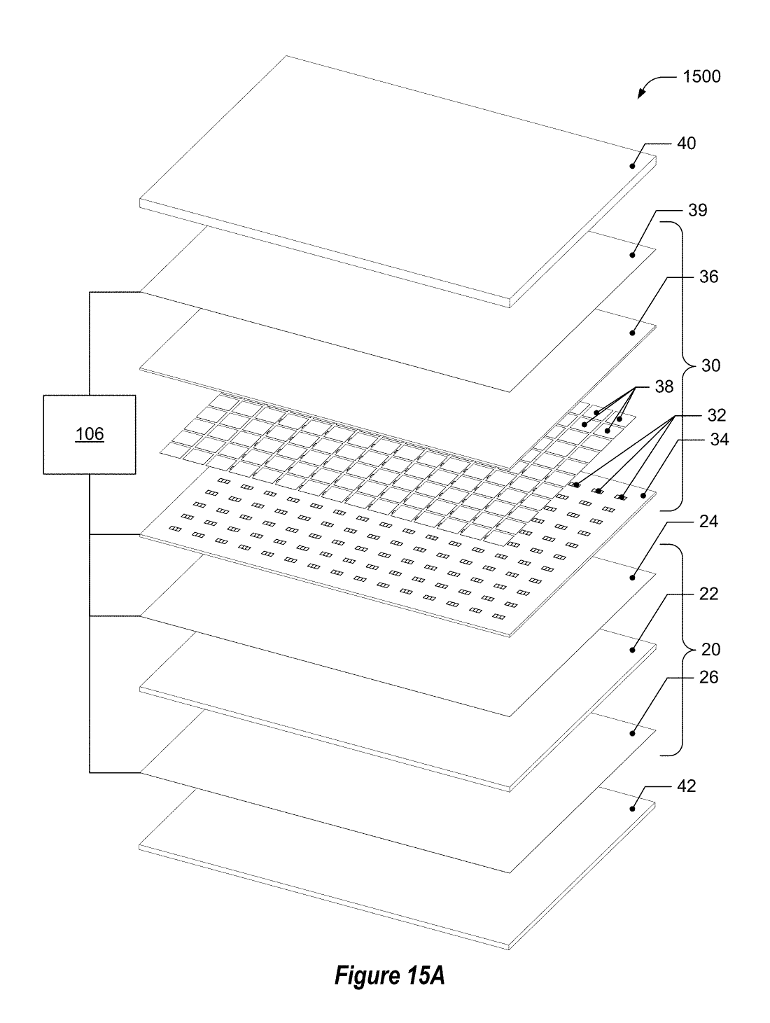

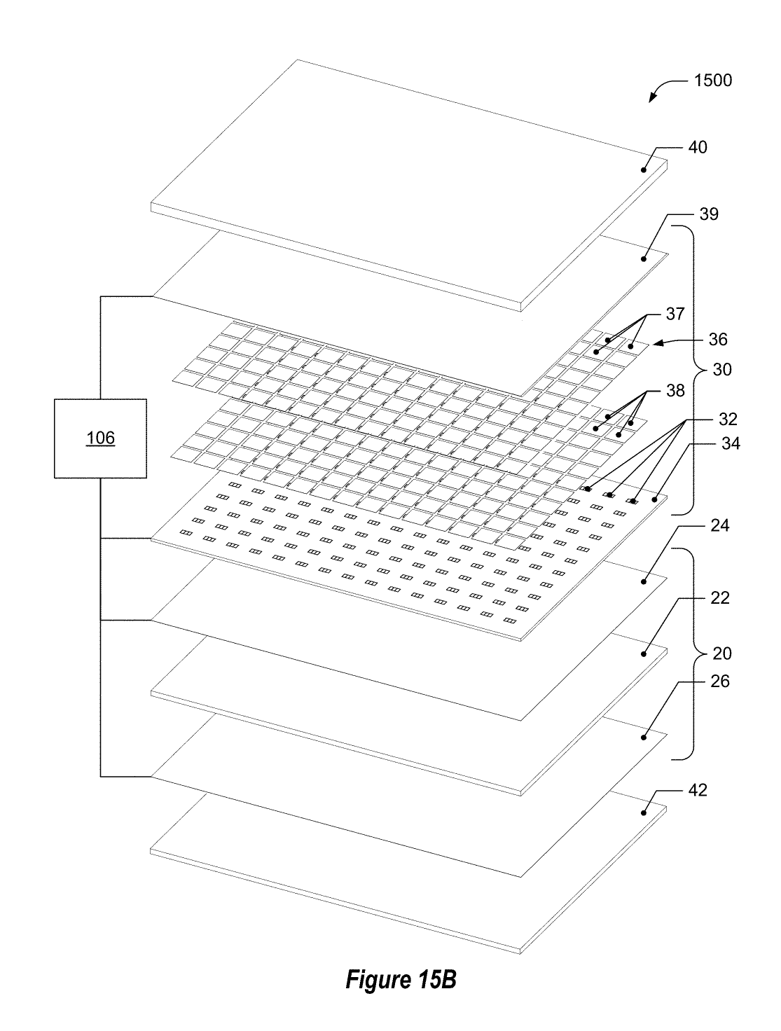

FIG. 15A shows an example of an exploded view of an ultrasonic sensor system. In this example, the ultrasonic sensor system 1500 includes an ultrasonic transmitter 20 and an ultrasonic receiver 30 under a platen 40. The ultrasonic transmitter 20 may include a substantially planar piezoelectric transmitter layer 22 and may be capable of functioning as a plane wave generator. Ultrasonic waves may be generated by applying a voltage to the piezoelectric layer to expand or contract the layer, depending upon the signal applied, thereby generating a plane wave. In this example, the control system 106 may be capable of causing a voltage that may be applied to the piezoelectric transmitter layer 22 via a first transmitter electrode 24 and a second transmitter electrode 26. In this fashion, an ultrasonic wave may be made by changing the thickness of the layer via a piezoelectric effect. This ultrasonic wave may travel towards a finger (or other object to be detected), passing through the platen 40. A portion of the wave not absorbed or transmitted by the object to be detected may be reflected so as to pass back through the platen 40 and be received by the ultrasonic receiver 30. The first and second transmitter electrodes 24 and 26 may be metallized electrodes, for example, metal layers that coat opposing sides of the piezoelectric transmitter layer 22.

The ultrasonic receiver 30 may include an array of sensor pixel circuits 32 disposed on a substrate 34, which also may be referred to as a backplane, and a piezoelectric receiver layer 36. In some implementations, each sensor pixel circuit 32 may include one or more TFT elements, electrical interconnect traces and, in some implementations, one or more additional circuit elements such as diodes, capacitors, and the like. Each sensor pixel circuit 32 may be configured to convert an electric charge generated in the piezoelectric receiver layer 36 proximate to the pixel circuit into an electrical signal. Each sensor pixel circuit 32 may include a pixel input electrode 38 that electrically couples the piezoelectric receiver layer 36 to the sensor pixel circuit 32.

In the illustrated implementation, a receiver bias electrode 39 is disposed on a side of the piezoelectric receiver layer 36 proximal to platen 40. The receiver bias electrode 39 may be a metallized electrode and may be grounded or biased to control which signals may be passed to the array of sensor pixel circuits 32. Ultrasonic energy that is reflected from the exposed (top) surface 42 of the platen 40 may be converted into localized electrical charges by the piezoelectric receiver layer 36. These localized charges may be collected by the pixel input electrodes 38 and passed on to the underlying sensor pixel circuits 32. The charges may be amplified or buffered by the sensor pixel circuits 32 and provided to the control system 106.

The control system 106 may be electrically connected (directly or indirectly) with the first transmitter electrode 24 and the second transmitter electrode 26, as well as with the receiver bias electrode 39 and the sensor pixel circuits 32 on the substrate 34. In some implementations, the control system 106 may operate substantially as described above. For example, the control system 106 may be capable of processing the amplified signals received from the sensor pixel circuits 32.

The control system 106 may be capable of controlling the ultrasonic transmitter 20 and/or the ultrasonic receiver 30 to obtain fingerprint image information, e.g., by obtaining fingerprint images. Whether or not the ultrasonic sensor system 1500 includes an ultrasonic transmitter 20, the control system 106 may be capable of controlling access to one or more devices based, at least in part, on the fingerprint image information. The ultrasonic sensor system 1500 (or an associated device) may include a memory system that includes one or more memory devices. In some implementations, the control system 106 may include at least a portion of the memory system. The control system 106 may be capable of capturing a fingerprint image and storing fingerprint image information in the memory system. In some implementations, the control system 106 may be capable of capturing a fingerprint image and storing fingerprint image information in the memory system even while maintaining the ultrasonic transmitter 20 in an “off” state.

In some implementations, the control system 106 may be capable of operating the ultrasonic sensor system 1500 in an ultrasonic imaging mode or a force-sensing mode. In some implementations, the control system may be capable of maintaining the ultrasonic transmitter 20 in an “off” state when operating the ultrasonic sensor system in a force-sensing mode. The ultrasonic receiver 30 may be capable of functioning as a force sensor when the ultrasonic sensor system 1500 is operating in the force-sensing mode. In some implementations, the control system 106 may be capable of controlling other devices, such as a display system, a communication system, etc. In some implementations, the control system 106 may be capable of operating the ultrasonic sensor system 1500 in a capacitive imaging mode.

The platen 40 may be any appropriate material that can be acoustically coupled to the receiver, with examples including plastic, ceramic, sapphire, metal and glass. In some implementations, the platen 40 may be a cover plate, e.g., a cover glass or a lens glass for a display. Particularly when the ultrasonic transmitter 20 is in use, fingerprint detection and imaging can be performed through relatively thick platens if desired, e.g., 3 mm and above. However, for implementations in which the ultrasonic receiver 30 is capable of imaging fingerprints in a force detection mode or a capacitance detection mode, a thinner and relatively more compliant platen 40 may be desirable. According to some such implementations, the platen 40 may include one or more polymers, such as one or more types of parylene, and may be substantially thinner. In some such implementations, the platen 40 may be tens of microns thick or even less than 10 microns thick.

Examples of piezoelectric materials that may be used to form the piezoelectric receiver layer 36 include piezoelectric polymers having appropriate acoustic properties, for example, an acoustic impedance between about 2.5 MRayls and 5 MRayls. Specific examples of piezoelectric materials that may be employed include ferroelectric polymers such as polyvinylidene fluoride (PVDF) and polyvinylidene fluoride-trifluoroethylene (PVDF-TrFE) copolymers. Examples of PVDF copolymers include 60:40 (molar percent) PVDF-TrFE, 70:30 PVDF-TrFE, 80:20 PVDF-TrFE, and 90:10 PVDR-TrFE. Other examples of piezoelectric materials that may be employed include polyvinylidene chloride (PVDC) homopolymers and copolymers, polytetrafluoroethylene (PTFE) homopolymers and copolymers, and diisopropylammonium bromide (DIPAB).

The thickness of each of the piezoelectric transmitter layer 22 and the piezoelectric receiver layer 36 may be selected so as to be suitable for generating and receiving ultrasonic waves. In one example, a PVDF piezoelectric transmitter layer 22 is approximately 28 μm thick and a PVDF-TrFE receiver layer 36 is approximately 12 μm thick. Example frequencies of the ultrasonic waves may be in the range of 5 MHz to 30 MHz, with wavelengths on the order of a millimeter or less.

FIG. 15B shows an exploded view of an alternative example of an ultrasonic sensor system. In this example, the piezoelectric receiver layer 36 has been formed into discrete elements 37. In the implementation shown in FIG. 15B, each of the discrete elements 37 corresponds with a single pixel input electrode 38 and a single sensor pixel circuit 32. However, in alternative implementations of the ultrasonic sensor system 1500, there is not necessarily a one-to-one correspondence between each of the discrete elements 37, a single pixel input electrode 38 and a single sensor pixel circuit 32. For example, in some implementations there may be multiple pixel input electrodes 38 and sensor pixel circuits 32 for a single discrete element 37.

FIGS. 15A and 15B show example arrangements of ultrasonic transmitters and receivers in an ultrasonic sensor system, with other arrangements possible. For example, in some implementations, the ultrasonic transmitter 20 may be above the ultrasonic receiver 30 and therefore closer to the object(s) 25 to be detected. In some implementations, the ultrasonic transmitter may be included with the ultrasonic receiver array (e.g., a single-layer transmitter and receiver). In some implementations, the ultrasonic sensor system 1500 may include an acoustic delay layer. For example, an acoustic delay layer may be incorporated into the ultrasonic sensor system 1500 between the ultrasonic transmitter 20 and the ultrasonic receiver 30. An acoustic delay layer may be employed to adjust the ultrasonic puke timing, and at the same time electrically insulate the ultrasonic receiver 30 from the ultrasonic transmitter 20. The acoustic delay layer may have a substantially uniform thickness, with the material used for the delay layer and/or the thickness of the delay layer selected to provide a desired delay in the time for reflected ultrasonic energy to reach the ultrasonic receiver 30. In doing so, the range of time during which an energy pulse that carries information about the object by virtue of having been reflected by the object may be made to arrive at the ultrasonic receiver 30 during a time range when it is unlikely that energy reflected from other parts of the ultrasonic sensor system 1500 is arriving at the ultrasonic receiver 30. In some implementations, the substrate 34 and/or the platen 40 may serve as an acoustic delay layer. In some implementations, a second platen 42 positioned below the sensor stack (e.g. a lower platen) may be included as shown in FIGS. 15A and 15B and as described above with respect to lower platen 302.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, e.g., a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also may be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium, such as a non-transitory medium. The processes of a method or algorithm disclosed herein may be implemented in a processor-executable software module which may reside on a computer-readable medium. Computer-readable media include both computer storage media and communication media including any medium that may be enabled to transfer a computer program from one place to another. Storage media may be any available media that may be accessed by a computer. By way of example, and not limitation, non-transitory media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection may be properly termed a computer-readable medium. Disk and disc, as used herein, includes compact disc (CD), laser disc, optical disc, digital versatile disc (DVD), floppy disk, and blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those having ordinary skill in the art, and the generic principles defined herein may be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “exemplary” is used exclusively herein, if at all, to mean “serving as an example, instance, or illustration.” Any implementation described herein as “exemplary” is not necessarily to be construed as preferred or advantageous over other implementations.

Certain features that are described in this specification in the context of separate implementations also may be implemented in combination in a single implementation. Conversely, various features that are described in the context of a single implementation also may be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, other implementations are within the scope of the following claims. In some cases, the actions recited in the claims may be performed in a different order and still achieve desirable results.

It will be understood that unless features in any of the particular described implementations are expressly identified as incompatible with one another or the surrounding context implies that they are mutually exclusive and not readily combinable in a complementary and/or supportive sense, the totality of this disclosure contemplates and envisions that specific features of those complementary implementations may be selectively combined to provide one or more comprehensive, but slightly different, technical solutions. It will therefore be further appreciated that the above description has been given by way of example only and that modifications in detail may be made within the scope of this disclosure.

1. An apparatus, comprising:
an ultrasonic receiver array;
an ultrasonic transmitter; and
a control system capable of:
controlling the ultrasonic transmitter to transmit first ultrasonic waves in a first direction and to simultaneously transmit second ultrasonic waves in a second direction that is opposite the first direction;
distinguishing first reflected waves from second reflected waves, the first reflected waves corresponding to reflections of the first ultrasonic waves that are received by the ultrasonic receiver array and the second reflected waves corresponding to reflections of the second ultrasonic waves that are received by the ultrasonic receiver array;
determining first image data corresponding to the first reflected waves; and
determining second image data corresponding to the second reflected waves.

2. The apparatus of claim 1, wherein the first reflected waves are received at the ultrasonic receiver array from a direction that is opposite a direction from which the second reflected waves are received.

3. The apparatus of claim 1, wherein the control system is capable of distinguishing the first reflected waves from the second reflected waves based on one or more factors selected from a list of factors consisting of a selected range-gate delay, frequency-dependent content of the first reflected waves and the second reflected waves, one or more image processing methods, and temperature data received from at least one temperature sensor.

4. The apparatus of claim 1, wherein the control system is capable of performing an authentication process, a liveness determination process or a pulse rate detection process according to at least one of the first image data and the second image data.

5. The apparatus of claim 4, wherein at least one of the first image data or the second image data includes fingerprint image data.

6. The apparatus of claim 1, wherein the first image data includes image data corresponding to a finger of a user and the second image data includes image data corresponding to a thumb of the user.

7. The apparatus of claim 1, wherein the control system is capable of:
detecting motion according to changes in at least one of the first image data or the second image data; and
producing a motion-detection signal that corresponds with a detected motion.

8. The apparatus of claim 7, further comprising a wireless interface, wherein the control system is capable of transmitting the motion-detection signal via the wireless interface.

9. A wearable device that includes the apparatus of claim 1.

10. The wearable device of claim 9, wherein the wearable device comprises a bracelet, an armband, a wristband, a ring, a headband or a patch.

11. The wearable device of claim 9, wherein the control system is capable of obtaining at least one of a tissue image or a bone image from a first side of the wearable device.

12. The wearable device of claim 11, wherein the control system is capable of obtaining a fingerprint image from a second side of the wearable device.

13. The wearable device of claim 12, wherein the control system is capable of performing an authentication process, a liveness determination process or a pulse rate detection process based, at least in part, on images obtained from the first side or the second side of the wearable device.

14. A smart card that includes the apparatus of claim 1, wherein the control system is capable of communication with a smart card reader.

15. The smart card of claim 14, wherein the control system is capable of performing an authentication process according to the first image data and the second image data, wherein at least one of the first image data or the second image data include fingerprint image data and wherein at least one of the first image data or the second image data include image data corresponding to a thumb.

16. A method of controlling a biometric sensor system, comprising:
controlling an ultrasonic transmitter to transmit first ultrasonic waves in a first direction and to simultaneously transmit second ultrasonic waves in a second direction that is opposite the first direction;
distinguishing first reflected waves from second reflected waves, the first reflected waves corresponding to reflections of the first ultrasonic waves that are received by an ultrasonic receiver array and the second reflected waves corresponding to reflections of the second ultrasonic waves that are received by the ultrasonic receiver array;
determining first image data corresponding to the first reflected waves; and
determining second image data corresponding to the second reflected waves.

17. The method of claim 16, wherein the first reflected waves are received at the ultrasonic receiver array from a direction that is opposite a direction from which the second reflected waves are received.

18. The method of claim 16, wherein distinguishing the first reflected waves from the second reflected waves is based on one or more factors selected from a list of factors consisting of a selected range-gate delay, frequency-dependent content of the first reflected waves and the second reflected waves, one or more image processing methods, and temperature data received from at least one temperature sensor of the biometric sensor system.

19. The method of claim 16, further comprising performing an authentication process, a liveness determination process or a pulse rate detection process according to at least one of the first image data and the second image data.

20. The method of claim 19, wherein at least one of the first image data or the second image data includes fingerprint image data.

21. A non-transitory medium having software stored thereon, the software including instructions for:
controlling an ultrasonic transmitter to transmit first ultrasonic waves in a first direction and to simultaneously transmit second ultrasonic waves in a second direction that is opposite the first direction; and
controlling a control system to:
distinguish first reflected waves from second reflected waves, the first reflected waves corresponding to reflections of the first ultrasonic waves that are received by an ultrasonic receiver array and the second reflected waves corresponding to reflections of the second ultrasonic waves that are received by the ultrasonic receiver array;
determine first image data corresponding to the first reflected waves; and
determine second image data corresponding to the second reflected waves.

22. The non-transitory medium of claim 21, wherein the first reflected waves are received at the ultrasonic receiver array from a direction that is opposite a direction from which the second reflected waves are received.

23. The non-transitory medium of claim 21, wherein the software includes instructions for controlling the control system to distinguish the first reflected waves from the second reflected waves based on one or more factors selected from a list of factors consisting of a selected range-gate delay, frequency-dependent content of the first reflected waves and the second reflected waves, one or more image processing methods, and temperature data received from at least one temperature sensor of the biometric sensor system.

24. The non-transitory medium of claim 21, wherein the software includes instructions for controlling the control system to perform an authentication process, a liveness determination process or a pulse rate detection process according to at least one of the first image data and the second image data.

25. The non-transitory medium of claim 24, wherein at least one of the first image data or the second image data includes fingerprint image data.

26. A smart card, comprising:
an ultrasonic receiver array;
an ultrasonic transmitter; and
a control system capable of:
controlling the ultrasonic transmitter to transmit first ultrasonic waves in a first direction and to simultaneously transmit second ultrasonic waves in a second direction that is opposite the first direction, wherein the first direction is towards a first side of the smart card and the second direction is towards a second side of the smart card;
distinguishing first reflected waves from second reflected waves, the first reflected waves corresponding to reflections of the first ultrasonic waves that are received by the ultrasonic receiver array and the second reflected waves corresponding to reflections of the second ultrasonic waves that are received by the ultrasonic receiver array;
determining first image data corresponding to the first reflected waves; and
determining second image data corresponding to the second reflected waves.

27. The smart card of claim 26, wherein the control system is capable of communication with a smart card reader.

28. The smart card of claim 26, wherein the control system is capable of performing an authentication process, a liveness determination process or a pulse rate detection process according to at least one of the first image data and the second image data.

29. The smart card of claim 28, wherein at least one of the first image data or the second image data includes fingerprint image data.

30. The smart card of claim 29, wherein at least one of the first image data or the second image data includes image data corresponding to a thumb.
